(19) United States
(12) Patent Application Publication
(10) Pub. No.: US 2025/0235216 A1
(43) Pub. Date: Jul. 24, 2025

(54) MEDICAL TOOL SYSTEM

(71) Applicant: Aesculap AG, Tuttlingen (DE)

(72) Inventors: Andre Buerk, Villingen-Schwenningen (DE); Thomas Hagen, Tuttlingen (DE); Simone Hermle, Villingen-Schwenningen (DE); Lilian Killinger, Ehningen (DE); Juergen Barth, Denkingen (DE); Roland-Alois Hoegerle, Tuttlingen (DE); Uwe Schaz, Neuhausen (DE)

(21) Appl. No.: 18/853,902

(22) PCT Filed: Mar. 24, 2023
(86) PCT No.: PCT/EP2023/057725

§ 371 Date: Oct. 3, 2024

(30) Foreign Application Priority Data
Apr. 4, 2022 (DE) 10 2022 107 972.4

(51) Int. Cl. A61B 17/16; A61B 17/00; A61B 90/00

(52) U.S. Cl. CPC A61B 17/162; A61B 17/1631; A61B 17/1633; A61B 90/03; A61B 2017/00477; A61B 2090/034

(57) ABSTRACT

A medical tool system includes a tool holder formed in a distal instrument portion adjoined by a proximal instrument portion. The proximal instrument portion is bendable relative to the distal instrument portion. A tool can be accommodated in the tool holder and pivoted relative to the tool holder. A first coupling device is arranged in the distal instrument portion. A second coupling device is arranged on the tool. In a coupling state, the second coupling device realizes a coupling between the tool and the distal shaft portion by operative engagement with the first coupling device to fix the tool in the distal shaft portion in the axial direction.

### CROSS-REFERENCE TO RELATED APPLICATION(S)

This application is the United States national stage entry of International Application No. PCT/EP2023/057725, filed on Mar. 24, 2023, and claims priority to German Application No. 10 2022 107 972.4, filed on Apr. 4, 2022. The contents of International Application No. PCT/EP2023/057725 and German Application No. 10 2022 107 972.4 are incorporated by reference herein in their entireties.

### FIELD

The present disclosure relates to a medical tool system comprising a medical, preferably shaft-shaped instrument having a distal shaft/instrument portion and a proximal shaft/instrument portion which can be bent relative to each other, and a medical tool of the shaft type, such as a drill, cutter, screwdriver, etc., which can be inserted into a tool chuck of the distal instrument portion in the axial direction, and is rotatable relative to the tool chuck but is supported in an axially fixed manner.

### BACKGROUND

Typically, such a (medical) tool has a distal tool head/effector and a tool shaft which is accommodated in the tool chuck. The tool head protrudes from the tool chuck and can be designed, for example, as a cutter, drill, cutting blade or the like. The tool chuck, which in this case holds the tool in a relatively rotatable manner, but does not itself rotate and is therefore also referred to as a tool holder for the sake of simplicity, is gripped (manually) by a user in order to guide the tool, preferably for surgery on a patient. Typically, the tool is rotated about its longitudinal axis relative to the tool holder. The rotational movement is transmitted to the tool shaft by a drive unit/drive train arranged in or on the tool holder.

In surgical technology, it is advantageous to be able to bend the tool head of a tool of a medical hand instrument relative to its tool shaft. In other words, a distal portion of the tool/tool shaft is to be bent relative to a proximal portion of the tool/tool shaft. This allows surgeries to be performed in very confined spaces, for example in case of surgeries on the spinal column.

In the field of surgical robots, instruments that allow the distal tool head to be bent relative to the rest of the tool shaft have been known for some time. This enables precise movement of the instruments in the tightest of spaces.

For example, milling tools with bendable tool shafts are known from the disclosures DE 10 2017 010 033 A1 and U.S. Pat. No. 10,178,998 B2. The distal tool head of the milling tool is bendable relative to the proximal milling tool shaft. The milling tool shaft in DE 10 2017 010 033 A1 is inserted into the tool holding portion of a medical instrument that can be gripped and guided by hand, and is secured axially against being pulled out of the tool holding portion via a push button that surrounds the milling tool shaft and is provided in a proximal region of the tool holding portion.

A disadvantage of medical tools in which the tool head is bendable relative to the tool shaft, as defined above, is that the set angle between the tool head and the tool shaft often changes undesirably under load.

For this reason, a tool holder of or for a medical hand instrument was developed, in which a distal instrument portion can be bent relative to a proximal instrument portion during operation and in which the angle between a longitudinal axis of the distal instrument portion and a longitudinal axis of the proximal instrument portion does not change even under load. In such a hand instrument, the tool shaft of the tool is inserted into the tool holder and can be bent together with the distal instrument portion with respect to the proximal instrument portion. In this way, the tool head can be bent relative to a proximal portion of the tool shaft together with the tool holder. In such a medical hand instrument, however, there is a risk that the tool will come out of the tool holder undesirably, especially under load.

The push-button safety device disclosed in DE 10 2017 010 033 A1 cannot be applied, or cannot be applied in a technically meaningful way, to the bendable tool holder of the medical hand instrument described above due to the different geometries of the respective tool holders.

### SUMMARY

The object of the disclosure is therefore to overcome the disadvantages of the prior art and to provide a medical shaft tool, a correspondingly designed medical instrument and a medical tool system, wherein the instrument has a bendable tool holder and a tool inserted therein and in which the tool does not undesirably detach from the tool holder. In particular, it is advantageous to couple the tool to the bendable tool holder in a particularly space-saving manner.

The basic idea of the present invention, for axially holding a tool in a tool holder of a medical instrument essentially consists in operatively connecting a rotary element arranged on the instrument via a slider to a coupling element acting radially on a tool shaft on/in the tool holder in a click-on manner such that, when the rotary element is rotated for a first predetermined time, the slider permits a release movement of the coupling element and, when the rotary element is rotated for a second predetermined time differently to the first rotation the slider blocks the release movement of the coupling element,.

If this functional basic principle is applied in a medical instrument which, as known from the aforementioned prior art, has two instrument portions that are rotatable relative to one another about a longitudinal axis of the tool shaft and are bent relative to one another with respect to the longitudinal axis of the shaft, the rotation mechanism can be used outside the range of rotation provided for bending the two instrument portions for actuating the aforementioned tool coupling.

In other words, a bendable instrument is conceivable, in which the distal instrument portion having a tool holder can be bent by a certain angle with respect to the proximal instrument portion, for which purpose the proximal instrument portion has to be rotated by a certain angle of rotation with respect to the distal instrument portion from a design position (e.g. with the two instrument portions aligned straight) in a specific direction of rotation. This allows using a rotational movement from the design position in the opposite direction for releasing the coupling element and thus axially decoupling the tool from the tool holder.

Specifically, to begin with, a medical shaft tool is proposed having a distal effector/tool head and a tool shaft that is provided and adapted to be inserted into the tool chuck of a medical instrument, preferably a hand instrument, in a rotatable but axially fixed manner. A coupling unit is provided for this purpose, which is or can be placed on the tool shaft and has the following components:
A ball or friction bearing for rotationally supporting a coupling unit housing on the tool shaft and
a latching/engagement geometry which is arranged or formed on an outer side of the coupling unit housing and defines an undercut acting in the axial direction, in particular in the proximal direction of the tool shaft.

This undercut serves as an axially acting point of attack of the coupling/snap-in element on the instrument side for axially holding the tool in the tool holder/tool chuck.

Consequently, also a medical instrument, in particular a hand instrument, is proposed, comprising a tool chuck/tool holder for selectively receiving a tool shaft, in particular as defined above, characterized by the following components:
an internal torque train provided and adapted to engage with the tool shaft in a torque transmitting engagement to drive the tool shaft,
an axially movable slider/latch/locking pin which is spring-preloaded in a first axial direction, preferably the proximal direction,
a latching engagement element, preferably a latching ball/locking ball as the aforementioned coupling element, which is in operative engagement with the slider/latch/locking pin such that the latching engagement element is shifted radially outwards into a release position (axially releasing the tool shaft) in a first axial position of the slider/latch/locking pin reached by the spring preload, and the latching engagement element is shifted radially inwards into a latching position (axially holding the tool shaft) in a second axial position of the slider/latch/locking pin reached against the spring preload.

Furthermore, an actuating housing portion rotatable relative to the slider/latch/locking pin about the tool holder axis is provided, in particular a sleeve-like actuating component including
an axial front or contact side, which faces the axial end portion of the slider/latch/locking pin on which the spring preload acts, so that the slider/latch/locking pin is pressed against the end face or contact side, and including
an axial recess or notch in the end face or contact side, into which the slider/latch/locking pin moves when the actuating housing portion is rotated for displacement thereof from its locking position into its release position.

This structure is particularly simple and insusceptible to manufacturing tolerances, contamination and/or wear.

Further preferably, the medical instrument has a distal instrument portion, which forms or comprises the tool chuck/tool holder and holds the slider/latch/locking pin in an axially displaceable manner and a proximal instrument portion, which forms or comprises the actuating housing portion, the distal and proximal instrument portions being in relative rotatable axial contact with one another at the end faces and the respective abutting end faces of the distal and proximal instrument portions being inclined to the longitudinal axis of the tool at an equal angle to each other greater than 0°, preferably 22.5°, such that in the event of relative rotation of the distal and proximal instrument portions, they are angularly displaced relative to one another in the axial direction. Advantageously, both the distal and proximal instrument portions are inclined by 22.5°, so that the angles add up to an adjustment angle of 45° at the maximum relative rotation to each other, more precisely at a relative rotation of 180° between the distal and proximal instrument portions.

In this case, the axial recess or notch in the front or contact side of the actuating housing portion can be positioned at such a circumferential position that when the distal and proximal instrument portions are angularly adjusted by rotating the actuating housing portion (in particular the entire distal instrument portion) within the range of rotation provided for this purpose it is impossible for the slider/latch/locking pin to retract into the axial recess or notch, however, when the distal and proximal instrument portions are angularly adjusted by rotating the actuating housing portion (in particular the entire distal instrument portion) outside the range of rotation provided for this purpose, it is possible for the slider/latch/locking pin to move into the axial recess or notch.

Accordingly, the disclosure also relates to a medical tool system with an instrument (possibly with integrated drive), which can be gripped in particular by a user for guiding the medical tool system and which has a distal instrument portion and a proximal instrument portion which can be bent relative to one another with respect to the longitudinal axis of the instrument by relative rotation about the longitudinal axis of the instrument. Furthermore, the medical tool system has a tool that is accommodated in the tool holder of the distal instrument portion and is pivoted relative to the tool holder. In the distal instrument portion a first coupling device (as defined above) is arranged and a second coupling device (as defined above) is provided on the tool. In a coupling state, the second coupling device implements a fixation of the tool in the axial direction by active engagement/interaction with the first coupling device.

Due to the interaction of the first coupling device in the distal instrument portion and the second coupling device on the tool, the tool is accommodated securely in the distal instrument portion in the axial direction. In this way, the tool cannot accidentally detach from the tool shaft. Especially when the medical tool system is in operation, it is of great advantage if the tool does not come loose from the tool holder in an undesired manner, so that injuries to a patient on whom the medical tool system is used can be avoided. Forces that can cause unwanted detachment of the tool from the tool holder are primarily tensile forces exerted on a distal end of the tool, in particular on its tool head. Hence, it is particularly advantageous if the first coupling device is arranged in the distal instrument portion (and not in the proximal instrument portion), as the coupling between tool and tool holder thus is located in the immediate vicinity of the location of the critical force and can counteract this (tensile) force. Accordingly, the coupling in the distal instrument portion between tool and tool holder is particularly effective and counteracts unintentional loosening of the tool from the tool holder particularly effectively.

In particular, the tool has a tool head and a tool shaft. The tool shaft is accommodated in the tool holder. The tool head protrudes from the distal instrument portion.

In this way, the tool head is exposed and can be applied particularly effectively on a patient.

Moreover, it is very useful if a third coupling device is further provided in the proximal instrument portion, and a fourth coupling device is provided on a proximal portion of the tool, in particular the tool shaft, which in a coupling state transmits a drive force/torque to the tool, in particular the tool shaft, by active engagement/interaction with the third coupling device.

The coupling in the proximal instrument portion (between the tool and the tool holder) thus serves to transmit a drive force, preferably within the proximal instrument portion, to the tool or the tool shaft. The coupling in the distal instrument portion (between the tool and the tool holder), however, acts as an axial lock for the tool in the tool holder.

It is particularly advantageous if the first coupling device has a locking ball and the second coupling device is an axial securing groove extending at least in sections in the circumferential direction of the tool, preferably completely circumferential in the circumferential direction, which is in engagement with the locking ball in the coupling state.

Such a coupling between the tool and the distal shaft section is particularly space-saving and can be accommodated in the smallest possible installation space.

Preferably, the axial securing groove has a semi-circular or arc-shaped cross-section. The locking ball is dimensioned in such a way that a portion of the locking ball is in full contact with the cross-sectional inner surface of the axial securing groove. In other words, the locking ball and the axial securing groove fit together exactly in terms of geometry and dimensions. In this way, the locking ball fits exactly into the axial locking groove and the coupling between the tool and the tool holder is implemented particularly effectively.

It is especially useful if the second coupling device, or more precisely the axial securing groove, is formed on a bearing housing of a roller bearing unit/housing of the coupling unit on the tool shaft side, which is fixed to the tool in the axial direction, i.e. in the direction of the longitudinal axis (of a distal portion) of the tool shaft, which is arranged in the distal instrument portion and pivots the tool relative to the distal instrument portion, in particular to the entire tool holder.

Pivoting of the tool in the tool holder makes sense, as the tool typically is pivoted relative to the tool holder when the medical tool system is in operation. Usually, the rotation is generated by the drive unit and transmitted to the tool shaft. In this respect, it is particularly effective in terms of precise and effective mounting if the roller bearing unit for pivoting the tool in the tool holder is provided in the distal instrument portion. If a roller bearing unit is provided on the tool, in particular on the tool shaft, it is particularly efficient and space-saving if the bearing housing of the roller bearing unit has the second coupling device, more specifically the axial securing groove. This is particularly relevant as the outer diameter of the tool holding shaft typically is 5.6 mm and the outer diameter of the roller bearing usually is 3 mm. The tool coupling, i.e. the first and second coupling device, has to be accommodated in the remaining, small installation space, minus the wall thickness of the tool holder.

Preferably, the roller bearing unit has at least one distal roller bearing, in particular a ball bearing, which faces the tool head, and one proximal roller bearing, in particular a ball bearing, spaced from the tool head in the axial direction and facing the proximal end of the tool shaft. Two ball bearings are able to support the tool shaft relative to the tool holder shaft in a particularly effective and low-friction pivotable manner. In addition, the bearing housing ensures safe and easy insertion of the tool in the tool holder shaft when the tool shaft is introduced into the tool holder shaft (coupling process).

In particular, the roller bearing unit is fixed to a distal portion of the tool shaft in the axial direction (of the tool shaft). To axially secure the roller bearing unit to the tool shaft, two stops may be provided that are arranged on the tool shaft, spaced apart from one another in the axial direction, preferably at least by the length of the roller bearing unit, between which the roller bearing unit is arranged. One of the two stops, in particular the one that is further away from the tool head than the other stop, can be connected to the tool shaft in one piece. The other stop is formed separately from the tool shaft and can be applied to the tool shaft and connected to it after the roller bearing unit has been arranged around the tool shaft. Alternatively, both stops can also be formed separately from the tool shaft. The stops, which are formed separately from the tool shaft, can be connected to the tool shaft in a form-fit or force-fit or material-fit manner.

In addition, the first coupling device preferably further comprises a locking pin/slider, which is fixed in the radial direction and can be moved in the axial direction between a coupling position/locking position, in which it holds the locking ball in the axial securing groove (in the radial direction) in order to realize the coupling state, and a release position, in which it does not hold the locking ball in the axial securing groove.

In other words, the distal end of the locking pin/slider in the coupling position is opposite the axial securing groove. In the release position, the distal end of the locking pin/slider is not opposite the axial securing groove. In the release position, the distal end of the locking pin is opposite a portion of the tool shaft (without recess) or a portion of the bearing housing of the roller bearing unit (without recess). The locking pin/slider advantageously allows the coupling between the tool and distal instrument portion not to unintentionally disengage by holding the locking ball in the axial securing groove. In the disengaged position, the locking pin prevents the locking ball from lying in the axial securing groove so that the tool can be taken from the tool holder shaft or can still be coupled to it.

It is also preferred that the locking pin/slider has a first locking ball receiving recess at its distal end, via which the locking pin in the coupling position holds the locking ball in the axial securing groove in the radial direction, and a second locking ball receiving recess which (when viewed in the axial direction) is closer to the distal end of the locking pin than the first locking ball receiving recess, and is provided further away from the tool or the bearing housing of the roller bearing unit than the first locking ball receiving recess in the radial direction and, in the release position, holds the locking ball on the tool in the radial direction, or on the bearing housing, but not in the axial securing groove.

The first and second locking ball receiving recesses can enclose the locking ball at least in portions and thus hold it optimally in the desired position. The first locking ball receiving recess is positioned on the locking pin in such a way that it can optimally interact with the axial securing groove in the coupling position of the locking pin. The second locking ball receiving recess is positioned on the locking pin such that it can optimally interact with the tool shaft or the bearing housing of the roller bearing unit in the release position of the locking pin/slider.

In particular, it is desirable that the two locking ball receiving recesses each have a semi-circular or circular arc-shaped cross-section. The locking ball is dimensioned such that at least a portion of the locking ball is in full contact with the cross-sectional inner surface of the first or second locking ball receiving recess. In other words, the locking ball and the two locking ball receiving recesses fit together exactly in terms of geometry and dimensions. In this way, the locking ball fits exactly into the first or second locking ball receiving recess and the adjustment of the coupling position or the release position of the locking pin/slider is implemented particularly effectively.

It is further conceivable that the proximal instrument portion has an edge at its distal end region, which holds the locking pin in the coupling position, and a locking pin receiving recess interrupting the edge, which receives a proximal end of the locking pin in the release position, as already indicated above.

In other words, the proximal end of the locking pin in the coupling position contacts the edge and is received in the locking pin receiving recess in the release position. The locking pin receiving recess allows the locking pin to move in the axial direction towards the proximal instrument portion. In this way, the distal end of the locking pin also moves in the axial direction away from the axial securing groove towards the proximal instrument portion. As soon as the proximal end of the locking pin is received in the locking pin receiving recess, the distal end of the locking pin is no longer opposite the axial securing groove and thus the locking pin has reached the release position. The locking pin receiving recess thus enables the realization of a non-coupling state between the tool and the tool holder. The edge, on the other hand, enables the coupling state to be maintained.

Preferably, only exactly one locking pin receiving recess interrupting the edge of the proximal instrument portion is provided.

In case more than one locking pin receiving recess are provided, there are also several positions in the circumferential direction in which the locking pin can be present in the release position. Multiple locking pin recesses would therefore impair the manageability and availability of the medical tool system without improving the coupling between the tool and the tool holder.

Preferably, the locking pin has a protrusion at its proximal end that is smaller in diameter than the rest of the locking pin and is dimensioned and shaped such that it is received in the locking pin receiving recess in the release position. In this way, the locking pin receiving recess can be dimensioned smaller than if the locking pin receiving recess had to accommodate the entire circumference of the locking pin instead of the (smaller) locking protrusion.

It is preferred that the edge at the distal end of the proximal instrument portion (apart from the locking pin receiving recess that interrupts it) is annular. The locking pin receiving recess interrupts this annular shape of the edge. If the edge is formed or shaped in such a manner the positioning of the locking pin in the axial direction can be changed together with a rotation of the distal shaft section relative to the proximal instrument portion between the release position and the coupling position, as the locking pin rotates together with the distal instrument portion.

It is useful if the first coupling device furthermore has a preload element, in particular a compression spring, preferably a spiral spring, which preloads the locking pin in the direction of the proximal shaft portion and thereby urges the locking pin, preferably the locking protrusion, into the locking pin receiving recess and thus into the release position.

The preload element ensures that the locking pin actually moves from the coupling position in the axial direction towards the proximal instrument portion into the locking pin receiving recess and thus into the release position when the locking pin and locking pin receiving recess are at the same height in the circumferential direction.

The preload element therefore likewise is part of the first coupling device. The coupling device thus has the locking ball, the locking pin and the preload element (spring) and is composed of these elements in particular.

Preferably, the maximum adjustable angle between the longitudinal shaft axis of the distal shaft portion and the longitudinal shaft axis of the proximal shaft portion is a 45° angle. Preferably, the predetermined angulation for tool unlocking is a (−)18° angle.

It is advantageous if the locking pin receiving recess has at least one beveled flank.

The beveled flank makes it easier for the locking pin to change between the release position and the coupling position. In other words, the beveled flank makes it easier for the locking pin to leave/emerge from the locking pin receiving recess.

Furthermore, it is conceivable that an outer circumferential surface of the bearing housing of the roller bearing unit, which is in abutting contact with an inner circumferential surface of the distal shaft portion and/or the distal shaft portion itself is/are provided with an adhesive surface coating, is roughened or has microgrooves, such a structure preferably being able to partially absorb a distally directed tensile force thus relieving the locking engagement element.

Compressive forces acting on the tool head when the medical tool system is in operation are transmitted by the roller bearing unit, in particular by the proximal roller bearing, directly (in the radial direction) to the distal instrument portion or its housing. In this way, the locking ball hardly has to absorb any compressive forces, but mainly has to counteract tensile forces in the axial direction. The special nature of the outer circumferential surface of the bearing housing of the roller bearing unit increases the transmission of tensile forces to the inner circumferential surface of the distal instrument portion and thus reduces the forces acting on the locking ball. Microgrooves in particular are perfectly suited for absorbing tensile forces. When using the medical tool system, the tool is not only loaded axially, but also radially. Due to the radial force component, the microgrooves are in active engagement with the outer circumferential surface of the bearing housing of the roller bearing unit and can thus reduce the load acting on the first coupling device, in particular the locking ball. This improves coupling between the tool and the tool receiving shaft and thus axial securing of the tool in the tool receiving shaft.

### BRIEF DESCRIPTION OF THE DRAWINGS

FIG. 1 is a longitudinal sectional view of a medical tool system in which a tool holder of the tool system is straight and a coupling state between a tool and a distal instrument portion is shown;

FIG. 2 is a longitudinal sectional view of the medical tool system, in which a distal instrument portion is bent at a 45° angle relative to a proximal instrument portion and a coupling state between the tool and the distal shaft portion is shown;

FIG. 3 is a perspective view of a tool with a roller bearing unit mounted on the tool shaft and having a second coupling device;

FIG. 4 is a longitudinal sectional view of a medical tool system in which a distal instrument portion is bent at a predetermined angle a relative to a proximal instrument portion and a non-coupling state between the tool and the distal shaft portion is shown;

FIG. 5 is a side view of a locking pin;

FIG. 6 is a perspective view of the distal end of the proximal instrument portion;

FIG. 7 is a top view of the distal end of the proximal instrument portion; and

FIG. 8 is a perspective view of the tool and the tool holder.

### DETAILED DESCRIPTION

Preferred aspects of the present disclosure are described below based on the associated figures.

In the following figures, the axial direction A and the radial direction R are marked to simplify understanding. Unless otherwise indicated, the axial direction A and the radial direction R each refer to a distal instrument portion with a tool holder of the medical tool system shown below.

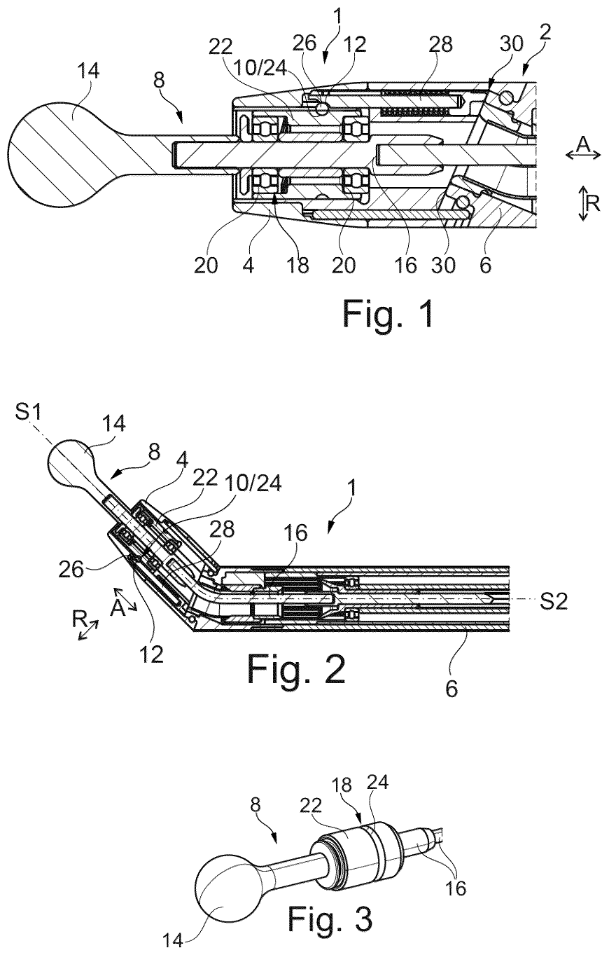

FIG. 1 is a longitudinal sectional view of a medical tool system 1, in which a tool holder 2 of the tool system 1 runs straight. The tool holder 2 is accommodated in a distal instrument portion 4, followed by a proximal instrument portion 6, which are bendable relative to each other. Furthermore, the medical tool system 1 has a tool 8, which is executed here as a cutter. The tool 8 is accommodated in the tool holder (tool chuck) 2 and is pivoted relative to the tool holder 2. A first coupling device 10 is disposed in the distal instrument portion 4. A second coupling device 12 is provided on the tool 8, which in a coupling state realizes a coupling between the tool 8 and the distal shaft portion 4 by interacting with the first coupling device 10 in order to fix the tool 8 in the distal shaft portion 4 in the axial direction A.

In FIG. 1, a longitudinal axis S1 of the distal instrument portion 4 and a longitudinal axis S2 of the proximal instrument portion 6 include an angle of 0°. In other words, the tool holder 2 extends straight with respect to the proximal instrument portion 6. In addition, FIG. 1 shows a coupling state between the tool 8 and the distal instrument portion 4. This means that the first coupling device 10, which is mounted in/on the distal instrument portion 4, and the second coupling device 12, which is provided on the tool 8, are in engagement or interact with each other. The tool 8 comprises a tool head/effector 14, more specifically a cutter head, and a tool shaft 16. The tool head 14 and the tool shaft 16 are connected to each other in a torque-proof manner.

From FIG. 1 there also is to be recognized that the tool shaft 16 is pivoted in the distal shaft portion 4 by means of a roller bearing unit 18. In the proximal instrument portion 6, a drive unit not shown here, is mounted/accommodated which applies a torque to the tool shaft 16. The tool shaft 16 rotates relative to the tool holder 2 due to this application of torque. The roller bearing unit 18 has at least one, here exactly two, roller bearings 20, more precisely ball bearings, which are spaced apart from one another in the axial direction A, but which alternatively can also be configured as friction bearings. The roller bearings 20 are accommodated in a bearing housing 22, which is part of the roller bearing unit 18. The second coupling device 12 is inserted in the bearing housing 22 and is therefore not provided directly on the tool 8. The roller bearing unit 18 and thus also the bearing housing 22 is arranged fixed to the tool shaft 16 in the axial direction A. Alternatively, however, it would also be conceivable that the second coupling device 12 is provided directly on the tool shaft 16.

The second coupling device 12 is configured as an axial securing groove 24 extending continuously in the circumferential direction of the bearing housing 22. The axial securing groove 24 preferably has a semicircular or circular segment-shaped cross-section. Accordingly, the first coupling device 10 provided in the distal instrument portion 4 has a locking ball 26. The diameter of the locking ball 26 is selected such that the locking ball 26 can be accommodated in the axial securing groove 24. Preferably, the axial locking groove 24 fully surrounds at least a portion of the locking ball 26 contacting the axial locking groove 24. In the coupling state, the locking ball 26 is held in the axial securing groove 24 on its side opposite the axial locking groove 24 by a distal end of a locking pin/slider 28. The locking pin 28 is part of the first coupling device 10. The locking pin 28 is fixed in the radial direction R. The locking pin 28 is arranged so as to be displaceable or movable in the axial direction in the distal shaft portion 4.

In the coupling state, the locking ball 26 thus is received in the axial securing groove 24 and is held in the axial securing groove 24 in the radial direction by the locking pin 28. This position of the locking pin 28 in the axial direction A is referred to as the coupling position. An edge 30 at the distal end of the proximal shaft portion 6 prevents movement of the locking pin 28 in the axial direction towards the proximal instrument portion 6.

FIG. 2 is a longitudinal sectional view of a medical tool system 1 in which the longitudinal shaft axis S1 of the distal instrument portion 4 is bent at a (+)45° angle relative to the longitudinal shaft axis S2 of the proximal instrument portion 6, and a coupling state between the tool 8 and the distal shaft portion 4 is shown. The 45° angle between the longitudinal shaft axis S1 of the distal instrument portion 4 and the longitudinal shaft axis S2 of the proximal instrument portion 6 is the maximum adjustable angle due to the selected angles (each corresponding to 22.5° to the longitudinal instrument axis) of the end surfaces of the distal instrument portion 4 and the proximal instrument portion 6, respectively, facing each other. The distal instrument portion 4 is bent relative to the proximal instrument portion 6 via a rotary mechanism that is not explained in detail here, the torque train between the drive and the tool shaft allowing this bending, as is implied in FIG. 2. It is to be recognized in FIG. 2 that the locking ball 26 is held in the axial locking groove 24 by the locking pin 28 in the radial direction R even when the tool holder 2 is bent in the maximum adjustable angular position with respect to the proximal instrument portion. In this way, the tool 8 is secured in the tool holder 2 by the engagement between the first coupling device 10 and the second coupling device 12, even in this angular position in axial direction A.

The torque strand towards the tool shaft 16, which is arranged in the transition region between the distal instrument portion 4 and the proximal instrument portion 6, is flexible. This flexible portion of the torque strand allows the distal portion of the tool shaft 16 with tool head 14 to be bent together with the distal instrument portion 4 relative to the torque strand in the proximal instrument portion. At the same time, the flexible portion of the torque strand is executed such that it can transmit a torque exerted on a proximal portion of the tool shaft 16 to the tool head 14.

FIG. 3 is a perspective view of the tool 8 with the roller bearing unit 18 mounted on the tool shaft 16. The axial securing groove 24, which is introduced in the bearing housing 22, clearly is to be seen here. The axial securing groove 24 extends over the entire circumference of the cylindrical bearing housing 22. The axial securing groove 24 is provided in a proximal half of the bearing housing 22 which faces the proximal end of the tool shaft 16.

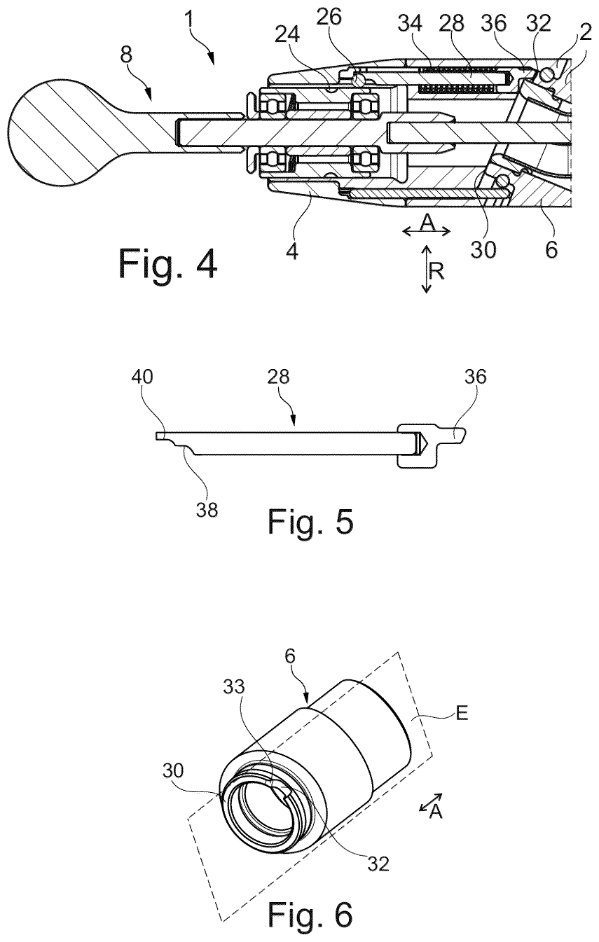

FIG. 4 is a longitudinal sectional view of the medical tool system 1, in which the distal instrument portion 4 is bent at a predetermined angle a relative to a proximal instrument portion 6, which is set when the proximal instrument portion is rotated by about −18°, i.e. against the direction of rotation, for “proper” bending of the instrument shaft portions, whereby a release state is achieved between the tool 8 and the distal instrument portion 4. It is to be discerned that the first coupling device 10 and the second coupling device 12 are not in operative engagement with each other in the release state. More precisely, the locking ball 26 is not received in the axial securing groove 24. Instead, it is held by the locking pin 28 against the outer circumferential surface of the bearing housing 22 in the radial direction R.

In order for the locking ball 26 to move out of the axial securing groove 24 into the release state, starting from the coupling state shown in FIG. 1, the locking pin 28 must move in the axial direction A towards the proximal instrument portion 6. In the coupling state, this is prevented by the edge 30 of the proximal instrument portion 6. However, the edge 30 is interrupted at one point by a locking pin receiving recess 32 (see also FIG. 6). When the relative rotation of the two instrument portions about the longitudinal axis S1 of the distal instrument portion 4 is beyond the range of rotation for “proper” bending, e.g. a (−)18° angle, then the locking pin 28 is positioned relative to the proximal instrument portion 6 such that, in the circumferential direction, it is at a level with the locking pin receiving recess 32. A preload element 34, which is a part of the first coupling device 10, urges the locking pin 28 in the axial direction A towards the proximal instrument portion 6. Thus, the preload element 34 pushes the locking pin 28, or more precisely its proximal end, which is formed as a locking protrusion 36, into the locking pin receiving recess 32. The position of the locking pin 28 when its locking protrusion 36 engages in the locking pin receiving recess 32 is referred to as the release position.

In the release position, the distal end of the locking pin 28 is no longer opposite the axial securing groove 24. This means that the locking ball 26 is not held in the axial securing groove 24 in the radial direction R. The tool 8 thus is no longer fixed relative to the distal shaft portion 4 in the axial direction A. If, starting from the coupling state, a tensile force (in axial direction A) is now applied to the distal end of the tool 8, the locking ball 26 is released from the axial securing groove 24. In this way, the tool 8 can be uncoupled from the tool holder 2.

FIG. 5 is a side view of the locking pin 28. At the proximal end of the locking pin 28, the locking protrusion 36 is to be recognized. The locking protrusion 36 is formed in the shape of a finger in order to be able to engage particularly securely and efficiently in the locking pin receiving recess 32 on the proximal instrument portion 6. At its distal end, the locking pin 28 has a first locking ball receiving recess 38 and a second locking ball receiving recess 40 on the side facing the tool 8. Both locking ball receiving recesses 38, 40 have a circular segment-shaped or hemispherical cross-section and are adapted to the diameter of the locking ball 26. More precisely, the two locking ball receiving recesses 38, 40 are dimensioned such that the locking ball receiving recesses 38, 40 fully surround the portion of the locking ball 26 that is in abutting contact with them.

The first locking ball receiving recess 38 is arranged on the locking pin 28 in such a way that it is opposite the axial securing groove 24 in the coupling position of the locking pin 28. In the coupling position, the first locking ball receiving recess 38 and the axial securing groove 24 therefore close around the locking ball 26 or are in contact with it. In the axial direction A, the second locking ball receiving recess 40 is located closer to the distal end of the locking pin 28 than the first locking ball receiving recess 38. The second locking ball receiving recess 40 is further away from the bearing housing 22 in the radial direction R than the first locking ball receiving recess 38. The second locking ball receiving recess 40 is in fact the locking ball receiving recess that, in the release position, holds the locking ball 26 against the outer circumferential surface of the bearing housing 22.

FIG. 6 is a perspective view of the distal end of the proximal instrument portion 6. The edge 30 protruding from the distal end of the proximal instrument portion 6 in the axial direction A (of the proximal instrument portion 6) is to be recognized. The edge 30 has an overall annular shape. The annular shape of the edge 30 is interrupted by the locking pin receiving recess 32. The end face of the distal end of the proximal instrument portion 6 is beveled at an angle of incidence with respect to the longitudinal axis of the instrument. More precisely, the end face of the distal end of the proximal shaft portion 6 here has an angle of incidence of 22.5° which, together with the 22.5° bevel of the opposite end edge of the distal instrument portion, defines the maximum adjustable angle between the longitudinal axis S1 of the distal instrument portion 4 and the longitudinal axis S2 of the proximal instrument portion 6. Furthermore, a plane E is to be discerned that bisects the proximal instrument portion 6 lengthwise. This plane E simultaneously marks an angulation of 0° or 45° of the distal instrument portion 4 to the proximal instrument portion 6. More precisely, the angle between the distal instrument portion 4 and the proximal instrument portion 6 is a 0° angle to 45° angle when the locking pin 28 (not shown here) is located in the plane E. The locking pin receiving recess 32 thus is positioned such that it does not lie in the plane E, or does not intersect it. It is to be discerned that the locking pin receiving recess 32 has at least one beveled flank 33, which facilitates the transition of the locking pin 28 between the release position and the coupling position or makes it possible at all in the first place.

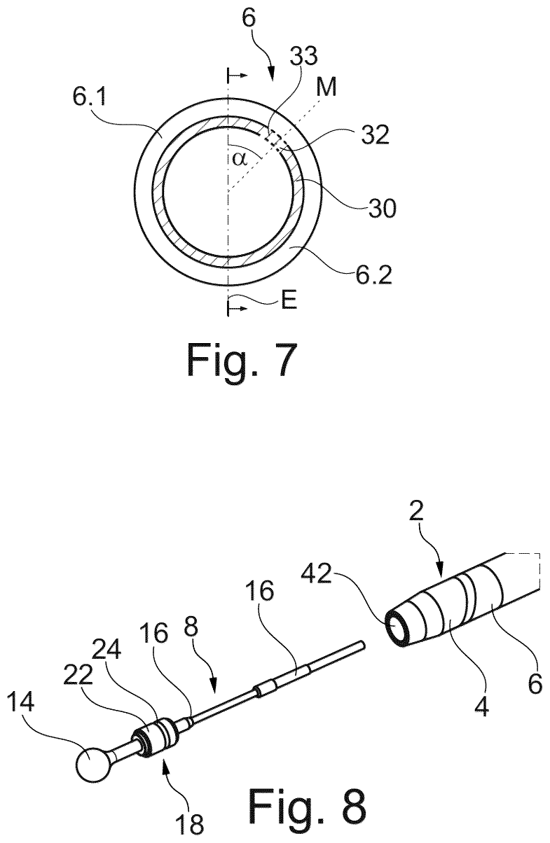

FIG. 7 is a top view of the distal end of the proximal instrument portion 6, showing the substantially annular edge 30. The ring shape of the edge 30 is interrupted by the circular segment-shaped locking pin receiving recess 32. Moreover, the plane E is to be recognized, which can only be represented here as a line. In addition, a central axis M is to be discerned, which divides the circular segment-shaped locking pin receiving recess 32 into two halves of equal size. The plane E and the center axis M include the predetermined angle of rotation, which is, for example, (−)18°. The plane E divides the proximal shaft portion 6 into two equally sized instrument portion halves 6.1 and 6.2. The locking pin receiving recess 32 is arranged in the right-hand half 6.2 of the instrument portion or half of the shaft portion shown here.

In case a user wishes to change the angle between the longitudinal axis S1 of the distal instrument portion 4 and the longitudinal axis S2 of the proximal instrument portion 6 from the 0° angle or from the maximum adjustable 45° angle while maintaining the coupling state, he/she should turn the distal instrument portion 4 relative to the proximal instrument portion 6 so that the locking pin 28 remains in the half 6.1 of the instrument portion. If the user wishes to end the coupling state, he/she should turn the distal instrument portion 4 relative to the proximal instrument portion 6 in exactly the other direction (i.e. in the minus direction), i.e. so that the locking pin 28 is moved into the instrument portion half 6.2. In doing so, the user must turn until the locking pin 28 or its locking protrusion 36 can engage in the locking pin receiving recess 32.

FIG. 8 is a perspective view of the tool 8 and the tool holder 2. To assemble the medical tool system 1, the tool 8 with its proximal end is introduced into an opening 42 at the distal end of the distal instrument portion 4. The opening 42 is large enough to accommodate the roller bearing unit 18 arranged on the tool shaft 16. The tool 8 is inserted into the tool holder 2 only far enough for the tool head 14 to protrude from the distal instrument portion 4.

To couple the tool 8 with the distal instrument portion 4, the predetermined angle of rotation between the distal instrument portion 4 and the proximal instrument portion 6 must be set, or the locking pin 28 (not shown here) has to be positioned in the release position. As soon as the tool 8 or its tool shaft 16 is properly accommodated in the tool holder 2, the angle of rotation between the distal instrument portion 4 and the proximal instrument portion 6 can be reset to the design position (straight alignment of the instrument). By rotating the distal instrument portion relative to the proximal instrument portion, the locking pin 28 is moved out of the locking projection receiving recess 32 via the beveled flank 33. The proximal end of the locking pin 28 or the locking protrusion 36 then is in contact with the edge 30. By the rotation of the distal instrument portion 4 relative to the proximal instrument portion 6 the locking pin 28 is moved (together with the distal instrument portion relative to the proximal instrument portion and thus) from the release position into the coupling position so that in this way the coupling state between the tool 8 and the tool holder 2 is set. In this coupling state, the tool 8 is then axially secured or fixed in the tool holder 2.

### List of reference signs

1 Medical tool system
2 Tool holder
4 Distal instrument portion
6 Proximal instrument portion
6.1 Left half of instrument portion
6.2 Right half of instrument portion
8 Tool
10 First coupling device
12 Second coupling device
14 Tool head
16 Tool shaft
18 Roller bearing unit/coupling unit
20 Roller bearing
22 Bearing housing
24 Axial securing groove
26 Locking ball
28 Locking pin/slider
30 Edge
32 Locking pin receiving recess
33 Beveled flank
34 Preload element/spring
36 Locking protrusion
38 First locking ball receiving recess
40 Second locking ball receiving recess
42 Opening
A Axial direction
E Plane
M Center axis
R Radial direction
S1 Longitudinal axis of distal instrument portion
S2 Longitudinal axis of proximal instrument portion

1.-15. (canceled)

16. A medical instrument comprising:
a tool chuck for selectively receiving a tool shaft of a medical shaft tool;
an internal torque train configured to engage with the tool shaft in torque transmitting engagement;
a slider or locking pin that is axially movable, the slider or locking pin being spring-preloaded in a first axial direction;
a locking engagement element in operative engagement with the slider or locking pin such that the locking engagement element is displaced radially outwards into a release position in a first axial position of the slider or locking pin reached through a spring preload, and, in a second axial position of the slider or locking pin reached against the spring preload, the locking engagement element is displaced radially inwards into a locking position; and
an actuating housing portion rotatable relative to the slider or locking pin about a longitudinal axis of the tool chuck,
the actuating housing portion having an axial front or contact side that faces an axial end portion of the slider or locking pin on which the spring preload acts, so that the slider or locking pin is pressed against the axial front or contact side, and
an axial recess or notch in the axial front or contact side, into which the slider or locking pin moves when the actuating housing portion is rotated for displacement from the locking position into the release position.

17. The medical instrument according to claim 16 further comprising:
a distal instrument portion that forms or comprises the tool chuck and holds the slider or locking pin in an axially displaceable manner; and
a proximal instrument portion that forms or comprises the actuating housing portion,
the distal instrument portion and the proximal instrument portion each having an end face and being in relative rotatable axial contact with one another at the end faces,
the end faces of the distal instrument portion and the proximal instrument portion being inclined at a mutually equal angle larger than 0° to a longitudinal axis of the medical instrument, such that, during relative rotation of the distal instrument portion and the proximal instrument portion, the distal instrument portion and the proximal instrument portion are angularly displaced relative to one another in a longitudinal direction of the medical instrument.

18. The medical instrument according to claim 17, wherein the axial recess or notch in the axial front or contact side of the actuating housing portion is placed on such a circumferential position that when the mutually equal angle of the distal instrument portion and the proximal instrument portion is shifted by turning the actuating housing portion within a range of rotation, retraction of the slider or locking pin into the axial recess or notch is prevented, and when the mutually equal angle of the distal instrument portion and the proximal instrument portion is shifted by turning the actuating housing portion outside of the range of rotation, retraction of the slider or locking pin into the axial recess or notch is permitted.

19. The medical instrument according to claim 18, wherein:
bending of the distal instrument portion and the proximal instrument portion is achieved by turning the actuating housing portion in a first direction of rotation, and
retraction of the slider or locking pin into the axial recess or notch is achieved by turning the actuating housing portion in a second direction of rotation opposite the first direction of rotation.

20. The medical instrument according to claim 19, wherein rotation of the actuating housing portion up to a retracted rotational position of the axial recess or notch with respect to the slider or locking pin is smaller than a maximum rotational range of the actuating housing portion provided for bending the distal instrument portion and the proximal instrument portion.

21. The medical instrument according to claim 16, wherein:
the slider or locking pin, in the locking position, is located opposite a locking geometry formed on the medical shaft tool and receiving the locking engagement element, and
the slider or locking pin, in the release position, is arranged with the axial end portion spaced apart from the locking geometry in a proximal direction.

22. The medical instrument according to claim 21, wherein the slider or locking pin further has a distal end portion and a first locking engagement element receiving recess at the distal end portion via which the slider or locking pin, in the locking position, holds the locking engagement element in the locking geometry in a radial direction and which, in the release position, releases the locking engagement element in the radial direction.

23. The medical instrument according to claim 22, wherein the slider or locking pin further has a second locking engagement element receiving recess at the distal end portion, which, viewed in an axial direction, is closer to the distal end portion than the first locking engagement element receiving recess, and is further away from the medical shaft tool in the radial direction than the first locking engagement element receiving recess and, in the release position, holds the locking engagement element in the radial direction on the medical shaft tool while not holding it in the locking geometry.

24. A medical tool system comprising:
the medical instrument according to claim 16; and
a medical shaft tool,
the medical shaft tool having a distal effector and a tool shaft provided and adapted to be inserted into the tool chuck of the medical instrument in a rotationally and axially fixed manner,
the medical shaft tool further comprising a coupling unit which is placeable on the tool shaft and comprises:
a roller or friction bearing for rotationally supporting a coupling unit housing on the tool shaft, and
a locking geometry arranged or formed on an outer circumferential surface of the coupling unit housing and defining an undercut acting in an axial direction.

25. The medical tool system according to claim 24, wherein:
the tool chuck forms a stop that projects radially inwardly, and
a proximal bearing unit on the tool shaft of the medical shaft tool axially abuts against the stop upon reaching an intended receiving position in the tool chuck, so as to provide an axial force transmission bridge for a proximally directed compressive force starting from the distal effector via the stop in the tool chuck.

26. The medical tool system according to claim 25, wherein the tool chuck has or forms a receiving sleeve having an inner diameter, the inner diameter adapted to an outer diameter of the coupling unit housing on a tool shaft side for a backlash-free insertion.

27. The medical tool system according to claim 24, wherein:
the medical instrument comprises a distal instrument portion that forms or comprises the tool chuck and holds the slider or locking pin in an axially displaceable manner,
an outer circumferential surface of the coupling unit housing is in abutting contact with the distal instrument portion, and
the outer circumferential surface of the coupling unit housing and/or the distal instrument portion is/are provided with an adhesive surface coating, is/are roughened or has/have microgrooves.

28. The medical tool system according to claim 24, wherein the roller or friction bearing has two axially spaced bearing units between which a spacer sleeve is arranged.

29. The medical tool system according to claim 28, wherein:
the roller or friction bearing comprises a roller bearing,
the two axially spaced bearing units each have an inner ring, an outer ring and a ball ring arranged between the inner ring and the outer ring,
the spacer sleeve is axially supported against the inner rings, and
the outer rings are inserted into the coupling unit housing.

30. The medical tool system according to claim 24, wherein the coupling unit housing has a cylindrical shape and the undercut is formed by a groove running around the outer circumferential surface of the coupling unit housing as the locking geometry.
